(19) United States
(12) Patent Application Publication
(10) Pub. No.: US 2004/0171175 A1
(43) Pub. Date: Sep. 2, 2004

(54) Process for conjugating biomolecules to hydrophobic membrane-incorporated molecules

(76) Inventors: Basil I. Swanson, Los Alamos, NM (US); Clifford J. Unkefer, Los Alamos, NM (US); Jurgen G. Schmidt, Los Alamos, NM (US); Scott M. Reed, Tesuque, NM (US); John E. Shively, Arcadia, CA (US); Lin Li, Monrovia, CA (US)

Correspondence Address: Bruce H. Cottrell, Los Alamos National Laboratory, LC/IP, MS A187, Los Alamos, NM, 87545, US

(21) Appl. No.: 10/377,515

(22) Filed: Feb. 28, 2003

(51) Int. Cl. G01N 33/543

(52) U.S. Cl. 436/518

(57) ABSTRACT

A process is provided of conjugating a recognition element such as a biomolecule to a hydrophobic multifunctional linker molecule by incorporating a multifunctional linker molecule including one or more anchoring groups, a reporter group and a reactive site thereon into a membrane, and, reacting the membrane including the incorporated multifunctional linker molecule with a pre-selected recognition element to form a covalently bound recognition element-multifunctional linker molecule-membrane assembly. Also, a chemical assembly suitable for subsequent covalent attachment of a recognition element is provided such assembly including a multifunctional linker molecule including one or more anchoring groups, a reporter group, and a hydrophilic spacer terminated by a reactive group capable of subsequent covalent bonding, the one or more anchoring groups incorporated in a membrane.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to a process for conjugating recognition elements such as biomolecules to hydrophobic membrane-incorporated molecules.

### BACKGROUND OF THE INVENTION

Trifunctional linker molecules anchorable into a membrane can be employed in sensors suitable for the ultra-sensitive detection of, e.g., bacterial and viral pathogens and cancer as previously described by Schmidt et al. in U.S. patent application Ser. No. 10/104,158, filed on Mar. 21, 2002. In one intermediate embodiment, such a trifunctional linker molecule includes three different functionalities, e.g., aliphatic chains to anchor the linker molecule into a bilayer membrane, a reporter material and a hydrophilic spacer terminated by a reactive group such as bromoacetamide. The general approach has been reaction or conjugation of the trifunctional moiety of this embodiment with a receptor or recognition element also terminated in a reactive group, e.g., a free SH group of a cysteine. The two reactive groups could be bound together, the linker molecule subsequently incorporated into a bilayer membrane, and the receptor or recognition element could then be available for binding with a target species at the surface of the bilayer membrane. These conjugation and membrane incorporation steps had been approached as separate reactions occurring within solution. Despite the various potential applications, the synthesis of such trifunctional linker molecules through this approach has been complicated by the extreme hydrophobicity of the membrane anchor groups (e.g., the aliphatic chains) thereby making both the conjugation step and membrane incorporation step difficult and sometimes even impossible to achieve. The hydrophobicity of the membrane anchor groups can make it difficult to keep the trifunctional linker molecule within an aqueous solution such that the trifunctional linker molecule precipitates (plates) out on surfaces. Thus, a need remains for an improved process for conjugation, i.e., formation of a chemical bond between, a recognition element such as a biomolecule and a hydrophobic molecule, especially an amphiphilic molecule including, e.g., a hydrophobic portion and a hydrophilic portion within the molecule.

The present invention describes a process of conjugating a recognition element such as a biomolecule to hydrophobic membrane-incorporated molecules.

Additionally, it is an object of the present invention to provide an intermediate chemical assembly suitable for subsequent attachment of a recognition element including a multifunctional linker molecule including one or more anchoring groups, a reporter group, and a hydrophilic spacer terminated by a reactive group, with the one or more anchoring groups incorporated in a membrane.

### SUMMARY OF THE INVENTION

In accordance with the purposes of the present invention, as embodied and broadly described herein, the present invention provides a process of conjugating a recognition element to a hydrophobic multifunctional linker molecule by incorporating a multifunctional linker molecule including one or more anchoring groups, a reporter group, and a reactive site thereon into a membrane, and, reacting the reactive site of the multifunctional linker molecule with a pre-selected recognition element, the multifunctional linker molecule present in the membrane, to form a covalently bound recognition element-multifunctional linker molecule-membrane assembly.

The present invention further provides a chemical assembly suitable for subsequent covalent attachment of a recognition element such chemical assembly including a multifunctional linker molecule including one or more anchoring groups, a reporter group, and a hydrophilic spacer terminated by a reactive group capable of subsequent covalent bonding, said one or more anchoring groups incorporated into a membrane.

### BRIEF DESCRIPTION OF THE DRAWINGS

FIG. 1 shows an exemplary reaction approach of the present invention.

FIG. 2 shows a schematic diagram of an apparatus for fluorescence correlation spectroscopy for measuring reactions in accordance with the present invention.

FIG. 3 shows a graph of an autocorrelation plot for green fluorescent protein in a stock solution alone and green fluorescent protein after reaction with a BODIPY®TRX-multifunctional linker molecule within a vesicle in accordance with example 2.

FIG. 4 shows a graph of an autocorrelation plot for green fluorescent protein in a stock solution alone and green fluorescent protein after reaction with a BODIPY®TMR-multifunctional linker molecule within a vesicle in accordance with example 4.

### DETAILED DESCRIPTION

The present invention concerns a process for conjugating recognition elements, e.g., biomolecules, to hydrophobic membrane-incorporated molecules. Initially, a multifunctional linker molecule including one or more of the anchoring groups can be incorporated into a membrane, e.g., a phospholipid bilayer membrane, in the form of vesicles (spheres) and the like, into immobilized membranes on a surface such as glass beads and the like or into cell membranes.

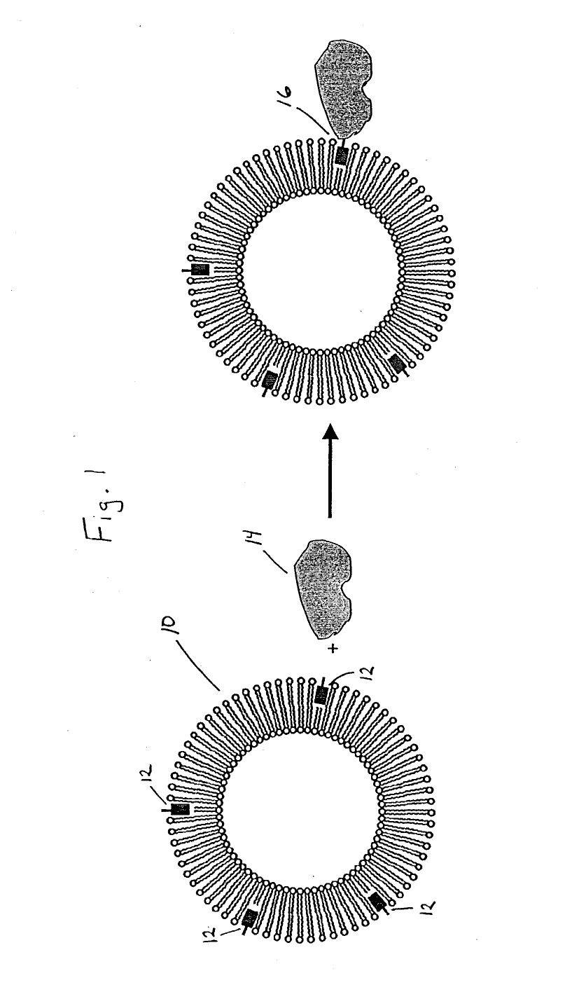

In FIG. 1, an exemplary reaction approach of the present invention is shown. Multifunctional linker molecules 12, which each include one or more anchoring groups, a reporter group and a reactive site thereon, are shown incorporated with vesicle 10. A pre-selected recognition element 14 is then reacted with the reactive site of a multifunctional linker molecule to yield a covalently bound recognition element-multifunctional linker molecule-membrane assembly 16.

Incorporation of a multifunctional linker molecule into a membrane can be readily accomplished through co-extrusion through filters with pores of different diameter or co-sonication as is well known to those skilled in the art.

Then, the resultant membrane assembly can be reacted with an appropriate receptor or recognition element. The process of the present invention can allow for better utilization of expensive reagents. In particular, a more precisely defined or pre-selected concentration of a recognition element (often an expensive biomolecule such as an antibody) can be used in the final reaction step. The present process also allows for better process control in that the multifunctional linker molecules including one or more of the anchoring groups can be dispersed more thoroughly throughout the membrane and avoid any self-aggregation that could often occur during aqueous reaction processes. By incorporation of the linker molecules into the membrane, the reactive site on such a multifunctional linker molecule can generally have a physical proximity away from the membrane surface due to the hydrophilicity of suitable spacer groups within the molecule. The reactive site on such a multifunctional linker molecule is typically hydrophilic in nature such that the multifunctional linker molecule may be referred to as an amphiphilic or amphiphatic molecule, i.e., a molecule with both hydrophobic and hydrophilic portions.

By “multifunctional” is meant molecules or species with at least three functionalities such as trifunctional or higher functionality molecules.

By “recognition element” is meant an element capable of recognizing and having a binding affinity for a specific target such as a biomolecule. Among such elements capable of recognizing and having a binding affinity for a specific target are biomolecules such as antibodies, peptides and mimetics thereof, sugars and mimetics thereof, oligosaccharides, proteins, nucleotides and analogs thereof, and receptor groups. An example of one class of receptor groups can be neuraminadase inhibitors such as described in U.S. patent application Ser. No. 09/699,225 for “Influenza Sensor” filed by Swanson et al. on Oct. 27, 2000, such description hereby incorporated by reference.

By “membrane” is generally meant vesicles, liposomes, immunoliposomes, supported bilayers where membrane layers are deposited upon a support surface, hybrid bilayers where a first layer is covalently attached to an oxide surface, tethered bilayers where a membrane molecule is covalently bonded to the oxide substrate, or bilayers cushioned by a polymer film. Supported membranes useful in the practice of the present invention are generally described by Sackmann, in “Supported Membranes: Scientific and Practical Applications”, Science, vol. 271, no. 5245, pp. 43-45, Jan. 5, 1996.

The reporter group generally yields an externally measurable output signal that can be correlated or assigned with a specific binding event. It can be generally chosen from among the following classes of commercially available detectable entities: fluorophores (e.g., BODIPY® dyes from Molecular Probes), radioisotopes and chelated derivatives thereof (e.g., technetium (Tc)), stable isotope labeled entities, magnetic particles or spin labeled carriers (e.g., for magnetic resonance imaging via nuclear magnetic resonance). The reporter groups can be hydrophobic in nature (e.g., BODIPY® dyes) or can be hydrophilic in nature (e.g., a fluorescein dye). Generally, it is preferred that the reporter group is attached separately upon the multifunctional linker molecule although less preferably the reporter group may be attached, e.g., upon an anchoring group attached upon the multifunctional linker molecule.

Optionally, other entities attached may be chosen for therapeutic purposes rather than imaging purposes and can include cytotoxic entities, carriers of radioisotopes and ligands for metals commonly used in treatment and diagnosis of tumor tissue.

The lipid components that can be used for the membrane layers in the present invention are generally described in the literature. Generally, these are phospholipids, such as, for example, phosphatidylcholines, phosphatidylethanolamines, phosphatidylglycerols, phosphatidylserines, phosphatidic acids, phosphatidylinositols or sphingolipids.

In one embodiment, the molecular assembly of a generic multifunctional linker molecule can represented by the general formula: (Re)(mA)CgSpRg where Cg is a trifunctional core, Re is a reporter group, mA is an anchoring group for mobile attachment into a membrane, Sp is a spacer group between the trifunctional core (Cg) and Rg, and Rg is a reactive or recognition element. Generally, these generic multifunctional linker molecules consist of a trifunctional core (Cg) derived, e.g., from amino acids such as lysine, homoserine, glutamic acid, serine, cysteine and the like. Attached to this trifunctional core are: (i) a reporter group (Re), e.g., a fluorophore, an isotopic label or another chemical and biochemical entity yielding an externally measurable output signal that can be correlated or assigned with a specific binding event; (ii) an anchoring group (mA), e.g., a long chain alkyl group; and, (iii) attachment arms carrying a receptor or recognition element (Rg) to bind to a target species. Such attachment arms can be composed of an amino acid side chain modified or extended by alkyl, ether, thioether or sulfone, phosphate and phosphonate, amide and amine containing spacers. One exemplary spacer group (Sp) is polyethylene glycol (PEG). These molecules can be used in assays towards binding events via the suitable reporter groups. After attaching a binding group (recognition group) on the linker and deprotection of the amino-end of the amino acid, a suitable reporter group, e.g., a BODIPY® fluorophore (a hydrophobic group) or fluorescein (a hydrophilic group), can be attached.

An exemplary reactive approach of the present invention is shown in FIG. 1. In general, it has been found that the reaction sequence of the present invention can provide a highly efficient reaction in terms of reactants and yield of the desired product especially in the case of antibodies as the reactive or recognition element. Also, as the reactive sequence of the present invention involves reaction with the appropriate receptor or recognition element only after the multifunctional linker molecule has already been incorporated or anchored into a membrane such as a bilayer membrane, the present process eliminates the need for a subsequent incorporation of a completely assembled multifunctional linker molecule into a membrane.

In a preferred embodiment of the present invention, the reactive site or reactive group on the multifunctional linker molecule is a haloacetamide, e.g., an iodoacetamide or bromoacetamide. Such reactive groups can be readily reacted with thiol (HS) bonds in selected recognition element molecules to form a covalent linkage. Alternatively, the reactive site may be a vinyl sulfone functionality or a phosphodiester linkage for reaction with a thiol bond in selected recognition element molecules to form a covalent linkage.

A water insoluble multifunctional linker molecule such as described in U.S. application Ser. No. 10/104,158, filed on Mar. 21, 2002 by Schmidt et al. for “Generic Membrane Anchoring System”, such description incorporated herein by reference, can be incorporated into vesicles in the following manner. The multifunctional linker molecule can be mixed with an organic solution (e.g., chloroform or methanol) of another lipid such as, e.g., 1-palmitoyl-2-oleoylphosphatidylcholine (POPC), at a pre-selected ratio. The mole ratio of the lipid to the multifunctional linker molecule can be varied, from about 10,000 to 1 to about 10 to 1, preferably from about 5000 to 1, to about 1000 to 1. A ratio of about 1000 to 1 has been demonstrated to provide satisfactory results.

After evaporation of the organic solvent under vacuum, the multifunctional linker molecule can then be brought into solution with the second lipid using standard techniques, such as rehydration followed by extrusion and/or sonication. These resultant vesicles can then be used directly or may be spread onto glass beads or glass surfaces. Incubation with 5 μm glass beads should form a phospholipid layer on the surface of the beads.

For example, preparation of vesicles containing a trifunctional linker may be accomplished in the following manner. A chloroform solution of 1-palmitoyl-2-oleoyl phosphatidylcholine may be dried into an even film on the bottom of a glass vial under nitrogen flow. Residual solvent may be removed under vacuum for several hours. A small amount of phosphate buffered saline (PBS) solution (about pH 7.4) may be added and the lipids allowed to rehydrate for about an hour at room temperature. Extrusion may be performed using an Avanti mini-extruder with a 100 nm pore polycarbonate filter. A portion of this solution may be added to glass beads. After incubating for a period of time, the beads may be washed repeatedly with the PBS solution. A methanol stock solution of BODIPY®-trifunctional linker molecule may then be added. After an incubation time, the beads may again be washed with the PBS solution. Fluorescence microscopy of such glass beads should show that the linker molecule including the fluorescent reporter dye is incorporated into the vesicles (membrane).

The present process provides several advantages over traditional solution phase chemistry. First, it provides a manner to conjugate a water-soluble recognition element (e.g., a biomolecule) to amphiphiles that are characterized as having an extremely hydrophobic portion. Further, it provides a manner to obtain a resultant chemical assembly of a multifunctional linker molecule including a further reactive site incorporated into a membrane. Further, it requires significantly less of the water-soluble recognition element to complete the conjugation reaction. As recognition elements such as antibodies and antibody fragments are often very expensive, it is advantageous to have a method of rapidly and efficiently conjugating such recognition elements to a desired platform. With this process, nearly any man-made receptor may be added to a multifunctional linker molecule previously incorporated into a membrane, e.g., a phospholipid bilayer membrane. Such a process should allow the mass manufacture of sensor elements having different receptors that can target different pre-selected molecules, e.g., proteins.

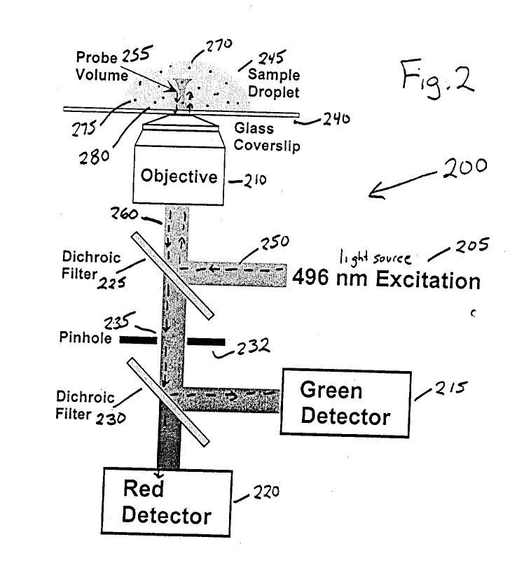

Fluorescence microscopy measurements were taken on an apparatus as shown in FIG. 2. Fluorescence correlation spectroscopy (FCS) is a standard technique commonly used in fluorescence-based detection assays and can be used to detect binding between the recognition element and the reactive site of the multifunctional linker molecule present within a membrane. FIG. 2 shows a schematic representation of a detection apparatus 200 for FCS measurement of a sample. Detection apparatus 200 includes a light source 205, an objective 210, a first detector 215, a second detector 220, a first dichroic filter 225, a second dicliroic filter 230, a support 232 having a pinhole 235 and a substrate 240.

In operation, detection apparatus 200 further includes an aqueous sample droplet 245, an excitation light beam 250, a probe volume 255, and an emission light beam 260. Detection apparatus 200 is typically an epi-fluorescence detection system, in which excitation light beam 250 travels through objective 210 to illuminate sample droplet 245 deposited on substrate 240. Substrate 240 is any transparent substrate, such as a glass microscope slide or cover slip that facilitates transmission of excitation light beam 250 and emission light beam 260. Emission light beam 260 from sample droplet 245 is subsequently collected and focused by objective 210. Sample droplet 245 further includes a plurality of membrane vesicles 270, targets 275 and library elements 280.

Light source 205 is any conventional light source, such as a specific wavelength laser or a mercury vapor arch burner, that provides excitation light beam 250 suitable for excitation of fluorophores in membrane vesicles 270 and target 275.

Objective 210 is any convenient converging lens, such as a 60×Nikon CFN Plan Apochromat, that focuses and transmits light. Probe volume 255 is the area of penetration of excitation light beam 250 from objective 210, and represents the area of sample droplet 245 under FCS analysis.

Detector 215 and detector 220 are conventional optical sensors, such as avalanche photodiodes (SPCM 200 PQ, Perkin Elmer Optoelectronics, Quebec, Canada) for detecting light of a specific wavelength, e.g., green and red light, respectively.

Dichroic filter 225 and dichroic filter 230 can be conventional longpass filters, such as XF2010 (Omega Optical) that reflect light shorter than a certain wavelength, and pass light longer than that wavelength. For example, dichroic filter 225 can reflect wavelengths below 500 nm (where excitation beam 250 is at 496 nm). This filter passes light above 500 nm, where fluorescence emission light beam 260 occurs as emitted fluorescence 210. The emission light beam 260 is further spectrally filtered by dichroic filter 230. Dichroic filter 230 can reflect emission light beam 260 below 550 nm and pass emission light beam 260 above 550 nm from sample droplet 245.

Pinhole 235 formed within support 232 can act as a spatial filter to block scattered light and penetration of “out of focus” emission light beam 260 from sample droplet 245 through objective 210. For example, “out of focus” emission light beam 260 is typically light that is not at the focal point of objective 210. Pinhole 235 effectively provides penetration of “in focus” emission light beam 260 to detector 215 and detector 220 via dichroic filter 230.

In an alternative embodiment, standard flow cytometry techniques may be used to detect interactions between biomolecules and to isolate positive binding events. In such an embodiment, membrane vesicles 270 can be coated onto spherical beads, such as glass beads or polystyrene beads using standard membrane coating procedures. These may be subsequently incubated with a solution that contains both target 220 and library element 240. Alternatively, target 220 or library element 240 may be attached to the reactive group. Cross correlation (i.e., co-localization) of the two different fluorescent signals (i.e., a green fluorescent signal and a red fluorescent signal co-localized to the same spherical bead within a flow stream) would be indicative of a positive binding event.

In a typical FCS measurement (i.e., autocorrelation or cross-correlation), fluorescence intensity is recorded over a time range from seconds to minutes. The time-dependent fluorescence intensity (I(t)) is then analyzed in terms of its temporal correlation function (G(τ)), which compares the fluorescence intensity at time t with the intensity at (t+τ), where τ is a variable interval averaged over all data points in a time series. Mathematical auto- or cross-correlation of the data uses the following general formula:

$$G ( τ ) = \frac{< δ {I}_{1} ( t ) δ {I}_{2} ( t + τ ) >}{< {I}_{1} ( t ) > < {I}_{2} ( t ) >}$$

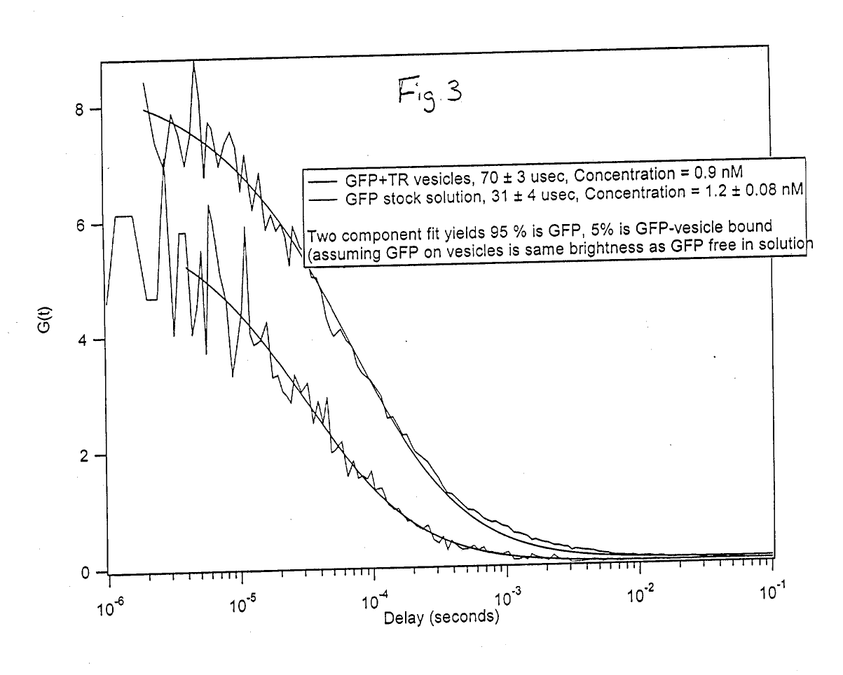

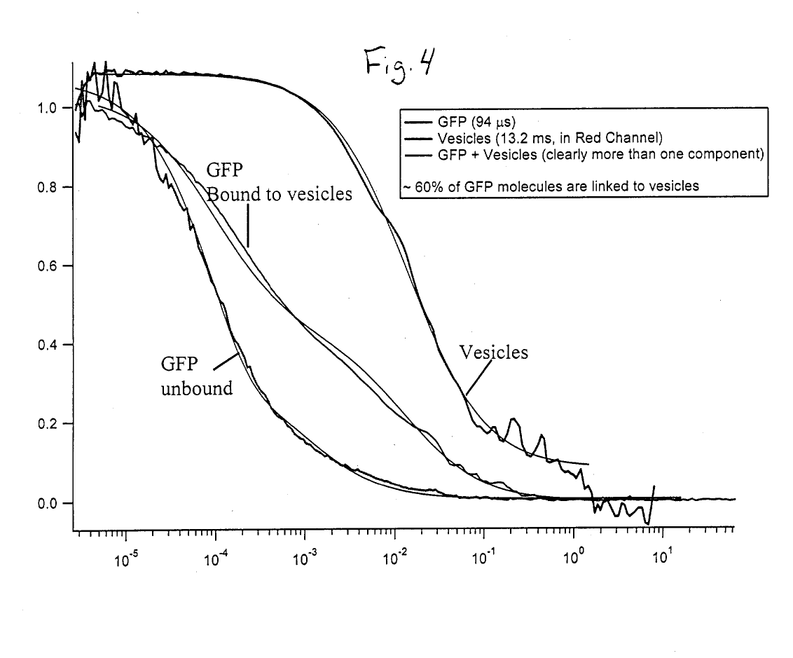

The autocorrelation function measures the time-dependent fluorescence intensity (I(t)) for a single fluorophore where I₁ and I₂ are fluorescence intensity signals at different delay times. The autocorrelation function provides quantitative data on the concentration and size (i.e., diffusion rates) of molecules in a sample. The autocorrelation function provides information on the interaction of two different molecules based on differences in their diffusion characteristics. For example, in FIGS. 3 and 4, the unbound material is the plot line to the left while the plot of the bound material is to the right.

The cross-correlation function measures time-dependent fluorescence intensities of two spectrally distinct fluorophores where I₁ and I₂ are fluorescence intensity signals for different wavelengths, e.g., a green fluorescent signal and a red fluorescent signal. The cross-correlation function provides quantitative information on the specific interactions between two molecules labeled with the spectrally distinct fluorophores. A cross-correlation signal is generated only when the two distinct fluorophores are detected in a single binding complex. Cross-correlation analysis eliminates background fluorescence from non-interacting molecules and can increase the sensitivity of detecting a binding event.

The present invention is more particularly described in the following examples which are intended as illustrative only, since numerous modifications and variations will be apparent to those skilled in the art.

### EXAMPLE A

Attachment of a fluorophore on an iodoacetamide PEG-Glu anchor was as follows.

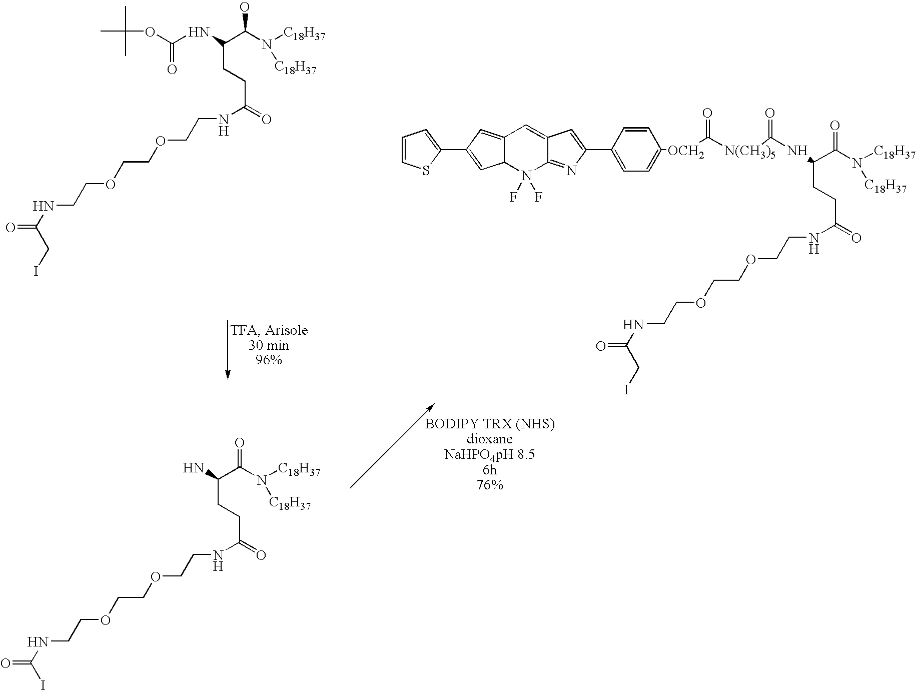

The Boc protecting group of a membrane anchor Glutamic acid iodoacetamide was removed in a 1:1:1 mixture of dichloromethane:TFA:anisole. The TFA salt of the anchor glutamic acid iodoacetamide was obtained in 98% yield after high vacuum (to 10 milliTorr (mT)) as a white salt and was sufficient in purity as determined by NMR for further reaction.

BODIPY®-TRX NHS ester (10 mg, 15.7 μmol) was added to the TFA salt of the Glutamic acid membrane anchor-PEG-iodoacetamide (16.7 mg, 15.7 μmol) in 1 mL 0.1 M phosphate buffer at pH 8.5. The residue in the BODIPY® vials was dissolved in 2 mL dioxane and added to the reaction. The reaction was stirred at room temperature under argon and shielded from light and monitored by thin layer chromatography on silica gel—60F254 (dichloromethane:methanol 5%) until complete conversion of the BODIPY® starting material (relative mobility to solvent front (Rf) 0.8) to one major product at Rf 0.2-0.3.

The reaction mixture was frozen in liquid nitrogen and lyophilized to dryness. The product was first purified by fast liquid chromatography on silica gel 60 at 6-8 psi using a gradient from 5 to 10% methanol in dichloromethane and after concentration on rotary evaporator the product was finally purified by high performance liquid chromatography on C18 reversed phase chromatography using a gradient from methanol to 10% dichloromethane. The product fractions were pooled then concentrated on rotary evaporator and dried on high vacuum to yield a blue solid (15.8 mg, 10.8 μmol, 68%), which should be stored at −70° C. and handled under argon and shielded from light.

### EXAMPLE B

Attachment of a fluorophore on a bromoacetamide PEG-Glu anchor was as shown schematically below in a similar manner to example 1. Generally, longer linking groups were added and particularly for this example, n was 12. The reporter (fluorescent) group was BODIPY®-TMR NHS ester.

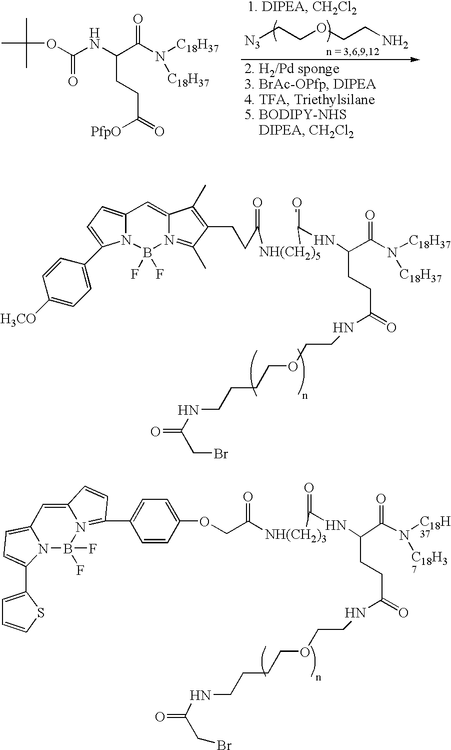

### EXAMPLE 1

Preparation of vesicles containing a trifunctional linker was as follows. A chloroform solution of 1-palmitoyl-2-oleoyl phosphatidylcholine (100 microliters, 5 mM) was dried into an even film on the bottom of a glass vial under nitrogen flow. A methanol stock solution of BODIPY®-trifunctional linker molecule (25 microliters, 20 μM), from example A, was added and methanol removed under nitrogen flow. Residual solvent was removed under vacuum for at least 4 hours. One mL of phosphate buffered saline (PBS) solution (pH 7.4) was added and the lipids were allowed to rehydrate for 1 hour at room temperature. Extrusion was performed using an Avanti Mini-Extruder (Avanti Polar Lipids Inc., Alabaster, Ala.) with a 100 nanometer (nm) pore polycarbonate filter. Alternatively, vesicles may be formed by sonication. After extrusion, vesicles were stored at 4° C. prior to subsequent use.

### EXAMPLE 2

Vesicle coupling of the trifunctional linker to a protein was as follows. An amount of green fluorescent protein (GFP), available from BD Biosciences Clontech, Palo Alto, Calif., was diluted in PBS solution at an equimolar concentration to the linker molecule-membrane assembly as prepared in Example 1. An equal volume of these two solutions were added and stirred at room temperature for 2 hours.

The resultant product was analyzed by FCS and yielded the plot in FIG. 3. The results demonstrated by the increase in the diffusion time indicate some binding occurred.

### EXAMPLE 3

Preparation of vesicles containing a trifunctional linker was as follows. A chloroform solution of 1-palmitoyl-2-oleoyl phosphatidylcholine (100 microliters, 5 mM) was dried into an even film on the bottom of a glass vial under nitrogen flow. A methanol stock solution of BODIPY®-trifunctional linker molecule (37 microliters, 27 μM), from example B, was added and methanol removed under nitrogen flow. Residual solvent was removed under vacuum for at least 4 hours. One mL of phosphate buffered saline (PBS) solution (pH 7.4) was added and the lipids were allowed to rehydrate for 1 hour at room temperature. Extrusion was performed using an Avanti Mini-Extruder with a 100 nm pore polycarbonate filter. Alternatively, vesicles may be formed by sonication. After extrusion, vesicles were stored at 4° C. prior to subsequent use.

### EXAMPLE 4

Vesicle coupling of the trifunctional linker to a protein was as follows. An amount of green fluorescent protein (GFP) was diluted in PBS solution at an equimolar concentration to the linker molecule-membrane assembly as prepared in Example 3. An equal volume of these two solutions were added and stirred at room temperature for 3 days.

The resultant product was analyzed by FCS and yielded the plot in FIG. 4. The results demonstrated by the increase in the diffusion time indicate about 60 percent binding occurred.

Although the present invention has been described with reference to specific details, it is not intended that such details should be regarded as limitations upon the scope of the invention, except as and to the extent that they are included in the accompanying claims.

What is claimed is:

1. A process of conjugating a recognition element to a hydrophobic multifunctional linker molecule comprising:
incorporating a multifunctional linker molecule including one or more anchoring groups, a reporter group and a reactive site thereon into a membrane;
reacting the reactive site of the multifunctional linker molecule with a pre-selected recognition element, said multifunctional linker molecule present in the membrane, to form a covalently bound recognition element-multifunctional linker molecule-membrane assembly.

2. The process of claim 1 wherein said incorporation is by co-extrusion.

3. The process of claim 1 wherein said incorporation is by co-sonication.

4. The process of claim 1 wherein said incorporation is by admixture of a multifunctional linker molecule in a solvent with a vesicle membrane.

5. The process of claim 1 wherein said recognition element is a natural or synthetic material selected from the group consisting of antibodies, peptides and mimetics thereof, sugars and mimetics thereof, oligosaccharides, proteins, nucleotides and analogs thereof and receptor groups.

6. The process of claim 1 wherein said membrane is selected from the group consisting of a bilayer membrane, a hybrid membrane, a tethered membrane, a vesicle, a membrane on a waveguide, a membrane on a solid support.

7. The process of claim 1 wherein said recognition element is a biomolecule.

8. The process of claim 1 wherein said multifunctional linker molecule is a trifunctional linker molecule.

9. The process of claim 1 wherein said recognition element is a natural or synthetic material.

10. The process of claim 9 wherein said recognition element is a neuraminadase inhibitor.

11. The process of claim 1 wherein said reporter group is hydrophilic.

12. The process of claim 1 wherein said reactive group is a haloacetamide group.

13. The process of claim 1 wherein said reporter group is separate from said one or more anchoring groups.

14. A chemical assembly suitable for subsequent covalent attachment of a recognition element comprising:
a multifunctional linker molecule including one or more anchoring groups, a reporter group, and a hydrophilic spacer terminated by a reactive group capable of subsequent covalent bonding, said one or more anchoring groups incorporated in a membrane.

15. The chemical assembly of claim 14 wherein said multifunctional linker molecule is a trifunctional linker molecule.

16. The assembly of claim 14 wherein said membrane is selected from the group consisting of a bilayer membrane, a hybrid membrane, a tethered membrane, a vesicle, a membrane on a waveguide, a membrane on a solid support.

17. The chemical assembly of claim 14 wherein said recognition element is a biomolecule.

18. The chemical assembly of claim 14 wherein said recognition element is a natural or synthetic material selected from the group consisting of antibodies, peptides and mimetics thereof, sugars and mimetics thereof, oligosaccharides, proteins, nucleotides and analogs thereof, receptor groups and recognition groups.

19. The chemical assembly of claim 14 wherein said reporter group is hydrophilic.

20. The chemical assembly of claim 14 wherein said reactive group is a haloacetamide group.

21. The chemical assembly of claim 14 wherein said reporter group is attached to said multifunctional linker molecule at a separate site from said attachment of said one or more anchoring groups.
